(19) United States
(12) Patent Application Publication
(10) Pub. No.: US 2008/0103346 A1
(43) Pub. Date: May 1, 2008

(54) Membrane Separation Of A Metathesis Reaction Mixture

(75) Inventors: Kenneth A. Burdett, Midland, MI (US); Baymond M. Collins, Midland, MI (US); Bob R. Maughon, Midland, MI (US); Michael L. Tulchinsky, Midland, MI (US)

Correspondence Address: The Dow Chemical Company, Intellectual Property Section, P.O. Box 1967, Midland, MI, 48641-1967, US

(73) Assignee: DOW GLOBAL TECHNOLOGIES, INC., Midland, MI (US)

(21) Appl. No.: 11/665,723

(22) PCT Filed: Oct. 14, 2005
(86) PCT No.: PCT/US05/36949

§ 371 Date: Jun. 18, 2007

Related U.S. Application Data
(60) Provisional application No. 60/620,998, filed on Oct. 21, 2004.

(51) Int. Cl. C07C 7/144

(52) U.S. Cl. 585/818

(57) ABSTRACT

A process for separating a homogeneous metathesis catalyst and, optionally, one or more homogeneous metathesis catalyst degradation products from a metathesis reaction mixture containing in addition to said metathesis catalyst and said metathesis catalyst degradation product(s), one or more olefin metathesis products, one or more unconverted reactant olefins, and optionally, a solvent. The process involves contacting the metathesis reaction mixture with a nanofiltration membrane, such as a polyimide nanofiltration membrane, so as to recover a permeate containing a substantial portion of the olefin reaction products, the unconverted reactant olefins, and optional solvent, and a retentate containing the metathesis catalyst, and optionally, metathesis catalyst degradation product(s). In another aspect, this invention pertains to a continuous metathesis reaction-catalyst separation process, preferably, conducted in a nanofiltration membrane reactor.

### CROSS REFERENCE STATEMENT

This application claims the benefit of U.S. Provisional Application No. 60/620,998, filed Oct. 21, 2004.

This invention was made with United States Government support under Award Number DE-FC36-01ID14213 (formerly Award Number DE-FC07-01ID14213) funded by the Department of Energy. The United States Government has certain rights in this invention.

### BACKGROUND OF THE INVENTION

In one aspect, this invention pertains to a separation of a homogeneous metathesis catalyst and, optionally, one or more homogeneous metathesis catalyst degradation products from a homogeneous metathesis reaction mixture.

In another aspect, this invention pertains to a continuous homogeneous metathesis process with continuous separation of a homogeneous metathesis catalyst, and optionally, one or more homogeneous metathesis catalyst degradation products from a homogeneous metathesis reaction mixture.

It is well known that olefinic products can be produced by homo-metathesis, cross-metathesis, ring-closing metathesis (RCM), and ring-opening metathesis polymerization (ROMP) as reported, for example, in the following patent publications: WO 96/04289, WO 02/076920, WO 03/093215, WO 2004/037754, and U.S. Pat. No. 6,156,692. Such processes generally involve contacting one or more reactant olefins in the presence of a metathesis catalyst to form at least one olefinic product that is different from the one or more reactant olefins. Homo-metathesis involves contacting one molecule of reactant olefin with a second molecule of the same olefin to prepare one or more olefinic products different from the reactant olefin. Cross-metathesis involves contacting a molecule of a first reactant olefin with a molecule of a second reactant olefin to prepare one or more olefinic products different from the reactant olefins. In RCM, two olefinic bonds within one molecule react to form a new olefin with ring-closing. In ROMP, a cyclic olefin is ring-opened and polymerized to form an open chain polymeric olefin. The metathesis catalyst typically comprises a catalytically active complex of a transition metal with at least one or more organic and, optionally, one or more inorganic ligands. Recently, cross-metathesis has been disclosed between a lower olefin, such as ethylene, with an unsaturated fatty acid or unsaturated fatty acid ester derived from seed oils to produce product olefins of intermediate chain length relative to the reactant olefins. More specifically, methyl oleate was disclosed, for example in WO 03/093215, to be cross-metathesized with ethylene in the presence of certain homogeneous ruthenium-ligand complex catalysts to form two terminal product olefins of intermediate chain length, namely, methyl 9-decenoate and 1-decene.

The aforementioned metathesis processes can be employed to up-grade the value of olefinic feedstocks, such as unsaturated feedstocks derived from seed oils, by converting such feedstocks into commercially desirable product olefins that find utility as monomers for polymers or as starting materials in the preparation of industrially useful organic chemicals.

When such processes employ a homogeneous metathesis catalyst, the subsequent separation of the catalyst from the reaction mixture becomes important. Incomplete separation of the transition metal of the complex catalyst is known to cause undesirable isomerization of the product olefin(s) during work-up or distillation. Moreover, since metathesis reactions are equilibrium processes, the equilibrium can be beneficially driven towards product(s) if olefin reactant(s) are added to the process while continuously removing a partially-converted reaction mixture from the process. In fact, it is desirable to push the equilibrium toward products as much as possible long before the catalyst deactivates. Without separation of the olefinic reaction products from the metathesis catalyst, inhibition of the catalyst by the product olefins may also occur. Such potential inhibition might require the use of lower conversions of reactant olefin(s) per pass with concomitant catalyst separation and recycle, if maximum catalyst productivity is to be achieved. For the above reasons, it is desirable to separate the homogeneous metathesis catalyst and, optionally, homogeneous metathesis catalyst degradation products, from the reaction mixture comprising, in addition to the metathesis catalyst and catalyst degradation products, one or more olefin metathesis products, one or more reactant olefins, and optionally a solvent.

The separation of a selective olefinic product in the metathesis of propene has been disclosed to be effected in a zeolite membrane reactor, as disclosed in “Application of a Zeolite Membrane Reactor in the Metathesis of Propene,” by Jolinde M. van de Graaf et al., Chemical Engineering Science, 54 (1999), pp. 1441-1445. The catalyst for the metathesis process is taught to be a heterogeneous rhenium oxide supported on alumina. The disclosure does not address the more difficult separation of a homogeneous metathesis catalyst from a metathesis reaction mixture.

US 2003/0135080 A1 discloses a homogeneous metathesis process for converting C₄ to C₁₀ olefins in a Fisher Tropsch-derived feedstock to C₆ to C₁₈ olefins using various ruthenium-based alkylidene catalysts in a metathesis process. It is only generally mentioned that the “catalyst may be separated from the product-catalyst mixture by short path distillation (SPD), membrane separation, immobilization on a suitable support carrier, phase separation or solvent extraction.”

WO 03/062253 discloses a metathesis process wherein a homogeneous metathesis catalyst is anchored to a dendritic material or inorganic carrier to render the catalyst essentially heterogeneous and therefore more easily removable from a metathesis reaction mixture by ultra-filtration.

Apparently, the prior art does not enable a process for separating a truly homogeneous metathesis catalyst and, optionally, homogeneous metathesis catalyst degradation products, absent a dendritic anchor or support, from a metathesis reaction mixture. Accordingly, a need exists in the art to discover and develop a membrane separation for such specialized use.

### SUMMARY OF THE INVENTION

In one aspect, this invention provides for a novel process of separating a homogeneous metathesis catalyst, and optionally, one or more homogeneous metathesis catalyst degradation products from a metathesis reaction mixture comprising, in addition to the homogeneous metathesis catalyst and the optional one or more homogeneous metathesis catalyst degradation products, one or more olefin metathesis products, one or more unconverted reactant olefins, and optionally, a solvent. The separation process of this invention comprises contacting said homogeneous metathesis reaction mixture with a nanofiltration membrane under conditions sufficient to allow a substantial portion of said one or more olefin metathesis products, said one or more unconverted reactant olefins, and optionally, said solvent to pass through the membrane as a permeate, while substantially rejecting said homogeneous metathesis catalyst and said optional one or more homogeneous metathesis degradation products as a retentate.

In a second aspect, this invention provides for a continuous metathesis and separation process comprising (a) contacting continuously one or more reactant olefins with a homogenous metathesis catalyst, optionally, in the presence of a solvent, in a reactor under reaction conditions sufficient to prepare a homogeneous metathesis reaction mixture comprising one or more metathesis olefin products that are different from the reactant olefins, one or more unconverted reactant olefins, the homogeneous metathesis catalyst, optionally, one or more homogeneous metathesis catalyst degradation products, and optionally the solvent; and (b) continuously contacting a portion of the homogeneous reaction mixture with a nanofiltration membrane under conditions sufficient to allow a substantial portion of said olefin metathesis products, said one or more unconverted olefin reactants, and said optional solvent to pass through the membrane as a permeate, while substantially rejecting said homogeneous metathesis catalyst and, optionally, said one or more homogeneous metathesis catalyst degradation products as a retentate; (c) recycling said retentate comprising said homogeneous metathesis catalyst to process step (a); and (d) separating said olefin metathesis products from the permeate and recycling the resulting permeate comprising said one or more unconverted reactant olefins and said optional solvent, essentially absent said olefin metathesis products, to process step (a).

The novel processes of this invention provide for efficient separation of a homogeneous metathesis catalyst and, optionally, one or more homogeneous metathesis catalyst degradation products from a reaction mixture comprising, in addition to said homogeneous metathesis catalyst and said optional homogeneous metathesis catalyst degradation products, one or more olefin metathesis products, one or more unconverted reactant olefins, and optionally, a solvent. The effective separation of the homogeneous metathesis catalyst and optional catalyst degradation products from the reaction mixture advantageously reduces the possibility of undesirable isomerizations of the olefin products during work-up or distillation. Moreover, since metathesis reactions are equilibrium processes, the separation process beneficially drives the metathesis equilibrium further in the direction of products, during the time that products are being removed and additional reactant olefins are being provided to the reactor. Advantageously, the separation of the metathesis catalyst and optional metathesis degradation products from the reaction mixture reduces the chance of catalyst inhibition by the reaction products. As a result, the conversion of olefinic reactant(s) and overall process productivity are enhanced prior to eventual catalyst deactivation. The separation of metathesis catalyst from the reaction process also allows for use of higher conversions of reactant olefin(s) per pass, thereby improving the efficiency of the metathesis reaction. As a further advantage, this invention avoids the need to anchor the metathesis catalyst to a dendritic material or ceramic support to render the catalyst heterogeneous. Thus, in contrast to the prior art, this invention provides for separation of a truly homogeneous metathesis catalyst from a metathesis reaction mixture. This invention is particularly suitable for separating homogeneous metathesis catalysts and, optionally, metathesis catalyst degradation products from reaction mixtures derived from the metathesis of seed oils (that is, unsaturated fatty acids or fatty acid esters), wherein the reactant and product olefins cannot be readily separated from the catalyst by vaporization, distillation or extraction.

### DETAILED DESCRIPTION OF THE INVENTION

This invention pertains to a novel process for the separation of a homogeneous metathesis catalyst and, optionally, one or more homogeneous metathesis catalyst degradation products from a metathesis reaction mixture. The invention finds particular adaptation to the separation of homogeneous metathesis catalysts and optional homogeneous metathesis catalyst degradation products from reaction mixtures derived from the metathesis of seed oils comprising unsaturated fatty acids or unsaturated fatty acid esters. The metathesis reaction mixture comprises, in addition to the homogeneous metathesis catalyst and, optionally, one or more homogeneous metathesis catalyst degradation products, one or more metathesis product olefins, one or more unconverted reactant olefins, and optionally, a solvent.

The process of this invention comprises contacting a metathesis reaction mixture comprising a homogeneous metathesis catalyst, optionally one of more homogeneous metathesis catalyst degradation products, one or more product olefins, one or more unconverted reactant olefins, and optionally a solvent, with a nanofiltration membrane under conditions sufficient to allow a substantial portion of said olefin products, said unconverted olefin reactants, and said optional solvent to pass through the membrane as a permeate, while substantially rejecting said homogeneous metathesis catalyst and said one or more optional homogeneous metathesis catalyst degradation products as a retentate.

In a preferred embodiment of this invention, the olefin reactant comprises an unsaturated fatty acid or unsaturated fatty acid ester derived from a seed oil. In another preferred embodiment of this invention, the olefin product comprises an α-olefin or an α,ω-unsaturated acid or ester.

In another aspect, this invention pertains to a continuous metathesis and separation process comprising (a) continuously contacting a reactant olefin and optionally a lower olefin, optionally in the presence of a solvent, with a homogeneous metathesis catalyst in a reactor under reaction conditions sufficient to prepare a homogeneous metathesis reaction mixture comprising the homogeneous metathesis catalyst, optionally one or more homogeneous metathesis catalyst degradation products, one or more product olefins that are different from the reactant olefins, one or more unconverted reactant olefins, and optionally a solvent; and (b) continuously contacting a portion of the homogeneous reaction mixture with a nanofiltration membrane under conditions sufficient to allow a substantial portion of said olefin metathesis products, said unconverted reactant olefins, and said optional solvent to pass through the membrane as a permeate, while substantially rejecting said homogeneous metathesis catalyst and said optional one or more homogeneous metathesis catalyst degradation products as a retentate; (c) recycling said retentate comprising said homogeneous metathesis catalyst to process step (a); (d) separating said olefin metathesis products from said permeate, and recycling the resulting permeate comprising said unconverted reactants and said optional solvent, essentially absent said olefin metathesis products, to process step (a).

In a preferred embodiment of the aforementioned invention, the continuous metathesis separation process is conducted in a membrane reactor containing a nanofiltration membrane.

In another aspect of the aforementioned processes, the reaction mixture further comprises one or more stabilizing ligands, as defined hereinafter. In yet another aspect of the aforementioned processes, the reaction mixture further comprises one or more non-ligand stabilizers, as defined hereinafter. If a stabilizing ligand and/or non-ligand stabilizer is present, these individually may be permeable or non-permeable with respect to the nanofiltration membrane, and therefore, may be found substantially in the permeate or retentate depending upon the specific membrane, stabilizing ligand, and/or non-ligand stabilizer.

In the inventions described herein, the metathesis reaction mixture can be obtained from any metathesis process, including for example, homo-metathesis processes between two reactant olefins of identical chemical structure; cross-metathesis processes between two reactant olefins of different chemical structure; ring-closing metathesis processes to form unsaturated ring compounds (RCM); and ring-opening polymerization processes (ROMP). Preferably, the metathesis reaction mixture is obtained from cross-metathesis by contacting a first reactant olefin with a second reactant olefin, typically a lower olefin, in the presence of a metathesis catalyst under reaction conditions sufficient to prepare one or more unsaturated products that are different from the reactant olefins. More preferably, the metathesis reaction mixture is obtained from cross-metathesis of an unsaturated fatty acid or unsaturated fatty acid ester derived from a seed oil with a lower olefin to form one or more product olefins having an intermediate chain length as compared with the reactant olefins. A most preferred metathesis process involves the cross-metathesis of a long-chain unsaturated olefin derived from a seed oil, such as methyl oleate or oleic acid, with a short chain olefin, preferably, a C₂₋₁₀ olefin, more preferably, ethylene, propylene, or decene, to form one or more intermediate-chain olefins, such as 1-decene or methyl 9-decenoate.

The generation of metathesis reaction mixtures via olefin metathesis processes is well-documented in the art, as noted for example, by K. J. Ivin and J. C. Mol, Olefin Metathesis and Metathesis Polymerization, Academic Press, San Diego, 1997, as well as by M. R. Buchmeiser, Chemical Reviews, 2000, 100, pp. 1565-1604; and by R. H. Grubbs, “Olefin Metathesis,” Tetrahedron, 2004, 60, pp. 7117-7140, all references being incorporated herein by reference.

There is no limitation on the source of the reactant olefin(s) that may be employed in the processes of this invention. Reactant olefins may be obtained from a variety of sources including conventional petroleum crackers, or Fisher Tropsch processes, or seed oils. The reactant olefin(s) may include any hydrocarbon or substituted hydrocarbon having at least one olefinic carbon-carbon double bond. Olefins containing more than one carbon-carbon double bond are also suitable. Preferred olefins contain from 2 to about 50 carbon atoms, more preferably, from 2 to about 30 carbon atoms. When two reactant olefins are employed, they can have the same or different chemical structures, and they can be each independently acyclic or cyclic. Each reactant olefin may be unsubstituted, or alternatively, substituted with any substituent that does not inhibit the desired metathesis process. Non-limiting examples of suitable substituents include alkyl moieties, preferably C₁₋₁₀ alkyl moieties, including for example, methyl, ethyl, propyl, butyl and higher homologues thereof; cycloalkyl moieties, preferably, C₄₋₈ cycloalkyl moieties, including for example, cyclopentyl and cyclohexyl; monocyclic aromatic moieties, preferably, C₆ aromatic moieties, that is, phenyl; arylalkyl moieties, preferably, C₇₋₁₆ arylalkyl moieties, including, for example, benzyl; and alkylaryl moieties, preferably, C₇₋₁₆ alkylaryl moieties, including, for example, tolyl, ethylphenyl, and xylyl; as well as ether, acyl, hydroxy, halo (preferably, chloro and bromo), nitro, carboxylic acid, ester, and amide moieties.

Non-limiting examples of suitable reactant olefins include ethylene, propylene, butylene, pentene, hexene, heptene, octene, nonene, decene, dodecene, cyclopentene, cyclohexene, cyclooctene, butadiene, octadiene, norbornene, dicyclopentadiene, cyclooctadiene, acrylamide, methyl acrylate; and unsaturated fatty acids, preferably, C₆₋₅₀ unsaturated fatty acids, such as 3-hexenoic (hydrosorbic), trans-2-heptenoic, 2-octenoic, 2-nonenoic, cis- and trans-4-decenoic, 9-decenoic (caproleic), 10-undecenoic (undecylenic), cis-4-dodecenoic (linderic), tridecenoic, cis-9-tetradecenoic (myristoleic), pentadecenoic, cis-9-hexadecenoic (cis-9-palmitoleic), trans-9-hexadecenoic (trans-9-palmitoleic), 9-heptadecenoic, cis-6-octadecenoic (petroselinic), trans-6-octadecenoic (petroselaidic), cis-9-octadecenoic (oleic), trans-9-octadecenoic (elaidic), cis-11-octadecenoic, trans-11-octadecenoic (vaccenic), cis-5-eicosenoic, cis-9-eicosenoic (gadoleic), cis-11-docosenoic (cetoleic), cis-13-docosenoic (erucic), trans-13-docosenoic (brassidic), cis-15-tetracosenoic (selacholeic), cis-17-hexacosenoic (ximenic), and cis-21-triacontenoic (lumequeic) acids, as well as 2,4-hexadienoic (sorbic), cis-9-cis-12-octadecadienoic (linoleic), cis-9-cis-12-cis-15-octadecatrienoic (linolenic), eleostearic, 12-hydroxy-cis-9-octadecenoic (ricinoleic), cis-5-docosenoic, cis-5,13-docosadienoic, and like acids.

Unsaturated fatty acid esters are also suitable metathesis reactants. The alcohol segment of the fatty acid ester can be any monohydric, dihydric, or polyhydric alcohol capable of condensation with an unsaturated fatty acid to form the ester. In seed oils the alcohol segment is glycerol, a trihydric alcohol. If desired, the glycerides can be converted via transesterification to fatty acid esters of lower alkanols, which may be more readily separated or suitable for downstream chemical processing. Typically, the alcohol used in transesterification contains at least one carbon atom. Typically, the alcohol contains less than about 15 carbon atoms, preferably, less than about 12 carbon atoms, more preferably, less than about 10 carbon atoms, and most preferably, less than about 8 carbon atoms. The carbon atoms in the alcohol segment may be arranged in a straight-chain or branched structure, and may be substituted with a variety of substituents, such as those previously disclosed hereinabove in connection with the reactant olefin, including the aforementioned alkyl, cycloalkyl, monocyclic aromatic, arylalkyl, alkylaryl, hydroxyl, halo, nitro, carboxylic acid, ether, ester, acyl, and amide substituents. Preferably, the alcohol segment of the unsaturated fatty acid ester is glycerol or a straight-chain or branched C₁₋₈ alkanol. Most preferably, the alcohol is a C₁₋₄ alkanol, suitable examples of which include methanol, ethanol, and propanol.

In a more preferred embodiment, one reactant olefin is a C₆₋₃₀ unsaturated fatty acid or unsaturated fatty acid ester, most preferably, oleic acid or an ester of oleic acid. If a second reactant is used, then the second reactant olefin is preferably a “lower olefin,” that is, a C₂₋₁₀ olefin, selected for example from ethylene, propylene, 1-butene, 2-butene, butadiene, pentenes, hexenes, heptenes, octenes, nonenes, decenes, and mixtures thereof. More preferably, the second reactant olefin is ethylene, propylene, or decene.

Metathesis process conditions are also well documented in the art. (See the references cited hereinabove.) The reactant olefin or mixture of olefins may be fed to the metathesis process in any operable quantity. When two different olefins are employed, one skilled in the art will know how to choose the relative amounts thereof, and if desired, how to minimize homo-metathesis reactions. Typically, the ratio of a first reactant olefin to a second reactant olefin is at least about 0.8/1. The following molar ratios may be used as a guideline for the metathesis of preferred long-chain unsaturated fatty acids or fatty acid esters with preferred lower olefins. Typically, the molar ratio of lower olefin to total unsaturated fatty acids or fatty acid esters is greater than about 0.8/1.0, preferably, greater than about 0.9/1.0. Typically, the molar ratio of lower olefin to total unsaturated fatty acids or fatty acid esters is less than about 5/1, and preferably, less than about 3/1. When the lower olefin is ethylene, homo-metathesis is not problematical, and the molar ratio of ethylene to unsaturated fatty acid or fatty acid ester can range up to about 20/1.0. More preferably, when ethylene is employed, the molar ratio is less than about 10/1.0.

The reactant olefin is typically provided to the metathesis process in a neat liquid phase without a solvent, because the use of a solvent may increase recycle requirements and costs. Optionally, however, a solvent may be employed, which may subsequently be recovered and recycled to the metathesis process. Non-limiting examples of suitable solvents include aromatic hydrocarbons, such as benzene, toluene, and xylenes; chlorinated aromatic hydrocarbons, preferably chlorinated benzenes, such as chlorobenzene and dichlorobenzene; alkanes, such as pentane, hexane, and cyclohexane; ethers, such as diethyl ether and tetrahydrofuran; and chlorinated alkanes, such as methylene chloride and chloroform. Any operable amount of solvent is acceptable. If a solvent is employed, then the concentration of each reactant olefin in the solvent is typically greater than about 0.05 M, preferably, greater than about 0.5 M, but typically, less than about the saturation concentration, and preferably, less than about 5.0 M.

Lower olefins, such as ethylene, propylene, and butenes, can be fed to the metathesis as an essentially pure gas or, optionally, diluted with a gaseous diluent. As the gaseous diluent, any substantially inert gas may be used, suitable examples of which include, without limitation, helium, neon, argon, nitrogen, and mixtures thereof. If a gaseous diluent is used, then the concentration of lower olefin in the diluent may suitably range from greater than about 5 mole percent, and preferably, greater than about 10 mole percent, to typically less than about 90 mole percent lower olefin, based on the total moles of lower olefin and gaseous diluent. Typically, oxygen is excluded from the metathesis process, so as to avoid undesirable side-reactions with the metathesis catalyst and its component parts (metal and ligands) as well as with reactant and product olefins.

As a further option, the metathesis reaction mixture may comprise a stabilizing ligand. The term “stabilizing ligand” embraces any molecule or ion capable of binding to the catalytic metal of the metathesis catalyst and capable of promoting catalyst stability in the metathesis process. Catalytic stability may be evidenced by a showing of increased catalyst activity or extended lifetime, as compared with the metathesis process in the absence of stabilizing ligand. The stabilizing ligand may be free and uncomplexed in the reaction mixture, or alternatively, bound to the transition metal of the catalyst for periods of time during the catalytic cycle. Non-limiting examples of stabilizing ligands include tri(alkyl)phosphines, such as tricyclohexylphosphine, tricyclopentylphosphine, and tributylphosphine; tri(aryl)phosphines, such as tri(phenyl)phosphine and tri(methylphenyl)phosphine; alkyldiarylphosphines, such as cyclohexyldiphenylphosphine; dialkylarylphosphines, such as dicyclohexylphenylphosphine; ethers, such as anisole; phosphine oxides, such as triphenylphosphine oxide; as well as phosphinites, phosphonites, phosphoramidites, pyridines, and any combination of the aforementioned compounds. Preferably, the stabilizing ligand is selected from the aforementioned phosphines, and more preferably, is tri(cyclohexyl)phosphine or tri(phenyl)phosphine. The quantity of stabilizing ligand can vary depending upon the specific catalyst employed and its specific ligand components. Typically, the molar ratio of stabilizing ligand to catalyst is greater than about 0.05/1, and preferably, greater than about 0.5/1. Typically, the molar ratio of stabilizing ligand to catalyst is less than about 2.0/1, and preferably, less than about 1.5/1. The stabilizing ligand, if used, may either permeate the nanofiltration membrane or remain with the retentate depending upon the specific stabilizing ligand and membrane in use.

In addition to the stabilizing ligand, the metathesis reaction mixture may also contain one or more non-ligand additives that also stabilize the catalyst and promote increased catalyst lifetime. The art discloses suitable non-ligand additives, their function, and their use in metathesis processes, as found for example, in WO 2004/056728 (Sasol), published Jul. 8, 2004. Such non-ligand additives may include aromatic compounds, such as phenolic and polyphenolic compounds. Preferably, the non-ligand additive is a phenolic compound, such as phenol or substituted derivatives of phenol. The non-ligand additive, if used, may either permeate the nanofiltration membrane or remain with the retentate depending upon the specific non-ligand additive and membrane in use.

The metathesis catalyst may comprise any catalyst that is capable of facilitating a metathesis process in homogeneous phase. Many metathesis catalysts are known in the art, representative examples of which are disclosed in WO 02/076920, WO 93/20111, U.S. Pat. No. 5,312,940, WO 96/04289; and in J. Kingsbury et al., Journal of the American Chemical Society, 121 (1999), 791-799; the aforementioned references being incorporated herein by reference. The preferred metathesis catalyst is a homogeneous catalyst comprising a transition metal selected from ruthenium, molybdenum, tungsten, rhenium, or a mixture thereof; more preferably, ruthenium, molybdenum, rhenium, or a mixture thereof; and most preferably, ruthenium. In addition to the transition metal, the preferred metathesis catalyst also comprises a carbene ligand and a plurality of anionic and/or electronically neutral ligands. In a more preferred embodiment, the catalyst comprises a transition metal, most preferably ruthenium, two anionic ligands, two electronically neutral ligands, and one carbene ligand; the catalyst, most preferably, having the structure of Formula I:

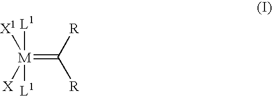

wherein M is ruthenium; each R is independently selected from hydrogen or a hydrocarbon selected from the group consisting of C₂-C₂₀ alkenyl, C₂-C₂₀ alkynyl, C₁-C₂₀ alkyl, C₆₋₂₅ aryl, C₁-C₂₀ carboxylate, C₂-C₂₀ alkoxy, C₂-C₂₀ alkenyloxy, C₂-C₂₀ alkynyloxy, C₆₋₂₅ aryloxy, C₂-C₂₀ alkoxycarbonyl, C₁-C₂₀ alkylthio, C₁-C₂₀ alkylsulfonyl, and C₁-C₂₀ alkylsulfinyl; X and X¹ are independently selected from any anionic ligand, preferably, chloride, bromide, and iodide; and each L¹ is independently selected from any neutral electron donor, preferably, phosphine, sulfonated phosphine, phosphite, phosphinite, phosphonite, arsine, stibine, ether, amine, amide, sulfoxide, carboxyl, nitrosyl, pyridine, and thioethers, most preferably, trialkylphosphine ligands where at least one of the alkyl groups is a secondary alkyl or a cycloalkyl.

Another more preferred metathesis catalyst comprises the chelated ruthenium complexes represented by Formula II:

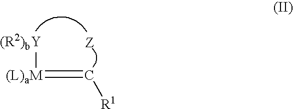

wherein M is Ru; each L is independently selected from neutral and anionic ligands in any combination that balances the bonding and charge requirements of M; a is an integer, preferably from 1 to about 4, which represents the total number of ligands L; R¹ is selected from hydrogen, straight-chain or branched alkyl, cycloalkyl, aryl, and substituted aryl radicals; Y is an electron donor group of an element from Group 15 or 16 of the Periodic Table, (as referenced by the IUPAC in Nomenclature of Inorganic Chemistry: Recommendations 1990, G. J. Leigh, Editor, Blackwell Scientific Publications, 1990); Y being more preferably O, S, N, or P; each R² is independently selected from hydrogen, alkyl, cycloalkyl, aryl, and substituted aryl radicals sufficient to satisfy the valency of Y, preferably such that Y is formally neutral; b is an integer, preferably 0 to about 2, representing the total number of R² radicals; and Z is an organic diradical that is bonded to both Y and the carbene carbon (C) so as to form a bidentate ligand, which ligand in connection with the M atom forms a ring of from about 4 to about 8 atoms. More preferably, each L in Formula II is independently selected from the group consisting of halides, most preferably, fluoride, chloride, bromide, and iodide; cyanide, thiocyanate, phosphines of the formula PR³₃, amines of the formula NR³₃, water and ethers of the formula OR³₂, thioethers of the formula SR³₂, and ligands having the Formulas III and IV hereinafter:

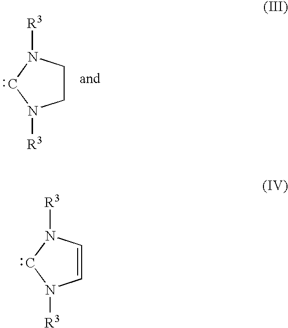

wherein each R³ in any of the aforementioned formulas is independently selected from the group consisting of hydrogen, alkyl, preferably, C₁₋₁₅ alkyl; cycloalkyl, preferably, C₃₋₈ cycloalkyl; aryl, preferably, C₆₋₁₅ aryl, and substituted aryl, preferably C₆₋₁₅ substituted aryl, radicals. Mixtures of any of the aforementioned ligands L may be employed in any given species of formula II. More preferably, R¹ in Formula II is selected from the group consisting of hydrogen, C₁₋₁₅ alkyl, C₃₋₈ cycloalkyl, and C₆₋₁₅ aryl radicals. More preferably, each R² is independently selected from the group consisting of C₁₋₁₅ alkyl, C₃₋₈ cycloalkyl, and C₆₋₁₅ aryl radicals. Preferably, Z is selected from the following diradicals: ethylene (V), vinylene (VI), phenylene (VII), substituted vinylenes (VIII), substituted phenylenes (IX), naphthylene (X), substituted naphthylenes (XI), piperazindiyl (XII), piperidiyl (XIII):

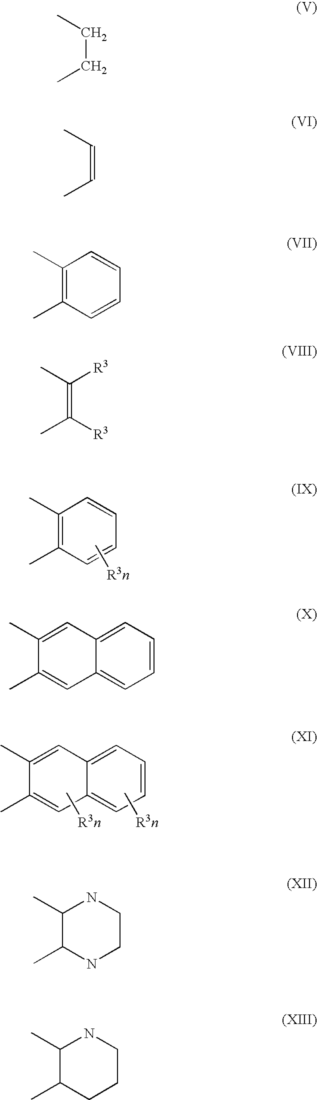

wherein each R³ may be, as noted above, selected from hydrogen, alkyl, preferably, C₁₋₁₅ alkyl; cycloalkyl, preferably, C₃₋₈ cycloalkyl; and aryl, preferably, C₆₋₁₅ substituted and unsubstituted aryl radicals; and wherein each n is an integer from 1 to about 4.

Non-limiting examples of suitable ruthenium catalysts include dichloro-3,3-diphenylvinylcarbene-bis(tricyclohexylphosphine)ruthenium (II), bis(tricyclohexyl-phosphine)benzylidene ruthenium dichloride, bis(tricyclohexylphosphine)benzylidene ruthenium dibromide, tricyclohexylphosphine[1,3-bis(2,4,6-trimethylphenyl)-4,5-dihydroimidazol-2-ylidene][benzylidene]ruthenium dichloride, tricyclohexylphosphine[1,3-bis(2,4,6-trimethylphenyl)-4,5-dihydroimidazol-2-ylidene][benzylidene]ruthenium dibromide, and tricyclohexylphosphine[1,3-bis(2,4,6-trimethylphenyl)-4,5-dihydroimidazol-2-ylidene][benzylidene]ruthenium diiodide. Non-limiting examples of suitable molybdenum, rhenium, and tungsten metathesis catalysts include 2,6-diisopropylphenylimido-neophylidenemolybdenum (VI) bis(hexafluoro-t-butoxide), as well as W(N-2,6-(i-Pr)₂Ph)(C-t-Bu)[OCMe(CF₃)₂]₂ and Re(C-t-Bu)(CH-t-Bu)[OCMe(CF₃)₂]₂ wherein “i-Pr” is isopropyl, “Ph” is phenyl, “t-Bu” is t-butyl, and “Me” is methyl.

Most preferably, the ruthenium metathesis catalyst is selected from the group consisting of dichloro-3,3-diphenylvinylcarbene-bis(tricyclohexylphosphine)ruthenium (II), bis(tricyclohexylphosphine)benzylidene ruthenium dichloride, tricyclohexylphosphine[1,3-bis(2,4,6-trimethylphenyl)-4,5-dihydroimidazol-2-ylidene][benzylidene]ruthenium (IV) dichloride, tricyclohexylphosphine[1,3-bis(2,4,6-trimethylphenyl)-4,5-dihydroimidazol-2-ylidene][benzylidene]ruthenium (IV) dibromide, tricyclohexylphosphine[1,3-bis(2,4,6-trimethylphenyl)-4,5-dihydroimidazol-2-ylidene][benzylidene]ruthenium (IV) diiodide.

The most preferred embodiment of Formula II is represented by Formula XIV:

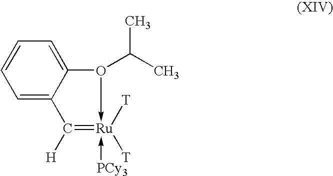

wherein each T is independently selected from Cl and Br, and PCy₃ represents tricyclohexylphosphine.

The term “metathesis catalyst degradation product” referred to hereinbefore shall include any derivative of the metathesis catalyst that is incapable of catalyzing the desired metathesis process. Such catalyst degradation products may include a molecule or ion derived from a ligand in the catalyst, such as a reaction by-product or oxidation product of such ligand, which may be uncomplexed or alternatively bound to metal. Such degradation products typically remain with the retentate.

Metathesis process conditions are well documented in the art, as noted in the above-cited references. Typical process conditions are summarized hereinbelow, but the inventions disclosed herein should not be bound or limited in any manner by the following statements. Other metathesis process conditions may be suitable depending upon the particular reactants and catalyst employed and upon the products targeted. Generally, the process is conducted at a temperature greater than about 0° C., preferably, greater than about 15° C., and more preferably, greater than about 25° C. Generally, the metathesis process is conducted at a temperature less than about 80° C., preferably, less than about 50° C., and more preferably, less than about 35° C. The total pressure, including reactant olefins and gaseous diluent, is typically greater than about 5 psig (34.5 kPa), preferably, greater than about 10 psig (68.9 kPa), and more preferably, greater than about 45 psig (310 kPa). Typically, the total pressure is less than about 500 psig (2,758 kPa), preferably, less than about 250 psig (1,723 kPa), and more preferably, less than about 100 psig (690 kPa).

If the metathesis process is conducted in a batch reactor, the ratio of moles of olefin feedstock to moles of metathesis catalyst is typically greater than about 10:1, preferably, greater than about 50:1, and more preferably, greater than about 100:1. The molar ratio of olefin feedstock to metathesis catalyst is typically less than about 10,000,000:1, preferably, less than about 1,000,000:1, and more preferably, less than about 500,000:1.

If the metathesis process is conducted in a continuous flow reactor, then the weight hourly space velocity, given in units of grams metathesis feedstock per gram catalyst per hour (h⁻¹) determines the relative quantity of reactant olefin(s) to catalyst employed, as well as the residence time of the olefin feedstock in the reactor. Accordingly, the weight hourly space velocity of the reactant olefin feedstock is typically greater than about 0.04 g per g catalyst per hour (h⁻¹), and preferably, greater than about 0.1 h⁻¹. The weight hourly space velocity of the olefin reactant feedstock is typically less than about 100 h⁻¹, and preferably, less than about 20 h⁻¹. When two olefins are employed, then the flows of the olefin reactants are typically adjusted to produce the desired ratio of first reactant olefin to second reactant olefin.

When the metathesis process is conducted as described hereinabove, then a metathesis reaction mixture is obtained that comprises a homogeneous metathesis catalyst, one or more product olefins that are different from the reactant olefin(s); one or more unconverted olefin reactants; optionally, a solvent; optionally, a stabilizing ligand; optionally, a non-ligand additive; and optionally, one or more metathesis catalyst degradation products. The product olefins may include monoolefins, diolefins, and polyolefins, and substituted derivatives thereof, providing at least one of these product olefins is different from the reactant olefin(s). The metathesis product olefins may include, for example, unsubstituted and substituted acyclic olefins, unsubstituted and substituted cyclic olefins, and conjugated 1,3-dienes. Suitable substituents have been named already in connection with the substituted reactant olefins. Preferably, the acyclic product olefin is a C₂₋₂₀ acyclic olefin. Preferably, the cyclic product olefin is a C₄₋₈ cyclic olefin. In a preferred embodiment of the invention, the metathesis product olefin is a C₂₋₂₀ α-olefin, such as 1-decene, or a C₂₋₂₀ α,ω-unsaturated ester or acid, such as methyl 9-decenoate.

A key feature in the process of this invention is the separation of the homogeneous metathesis catalyst and, optionally, one or more homogeneous metathesis catalyst degradation products from the metathesis reaction mixture, the separation being effected by membrane separation techniques, more specifically, by use of a nanofiltration membrane. The membrane used in the process of the present invention is characterized as a solid material containing discrete pores that provide pathways through the solid. The term “nanofiltration membrane” generally refers to a group of membranes having a pore size ranging from about 1 nanometer (10 Angstroms) up to about 100 nanometers (1,000 Angstroms) in diameter or critical dimension (in those instances wherein the pore is not exactly circular in cross-section). It should be understood that typically the membrane will possess a distribution of pore sizes, which may comprise a narrow distribution or a broad distribution depending upon the specific membrane and its method of preparation. Nanofiltration membranes can be obtained from commercial sources, such as, MET Ltd., Imperial College, London, UK, or from KOCH Membrane Systems, Inc. With use of such membranes, the metathesis catalyst and optional metathesis catalyst degradation products are substantially retained as a retentate, while the organic components of the metathesis reaction mixture including product olefins, unconverted reactant olefins, and optional solvent are substantially passed through the membrane filter as a permeate.

Preferably, the pore size of the nanofiltration membrane suitable for this invention is reflected in terms of a “molecular weight cutoff” or “MWCO,” that is, the molecular weight beyond which a material of higher molecular weight substantially cannot pass through the membrane and below which a material of lower molecular weight substantially passes through the membrane. Suitably, the MWCO is lower than the molecular weight of the metathesis catalyst; or alternatively, the mean pore size of the membrane is less than the molecular diameter of the metathesis catalyst. In the process of this invention, the MWCO is preferably less than about 800 Daltons (atomic mass units), and more preferably, less than about 500 Daltons. Preferably, the MWCO is greater than about 200 Daltons, and more preferably, greater than about 400 Daltons. In addition, the nanofiltration membrane may be characterized by a “flux” or “permeability,” which is defined as the quantity of fluid typically passing through the membrane per unit area of membrane per hour. The nanofiltration membrane preferred for the process of this invention has a flux or permeability greater than about 1 liter per meter² per hour (L/m²-h); preferably, a flux or permeability greater than about 3 L/m²-h; and more preferably, a flux or permeability greater than about 5 L/m²-h. Typically, the nanofiltration membrane preferred for the process of this invention has a flux or permeability less than about 100 L/m²-h. Additionally, the membrane is preferably chemically resistant to the catalyst and organic compounds in the metathesis reaction mixture, including the reactant olefins, the product olefins, any stabilizing ligand, any non-ligand additive, and the organic solvent in use. The term “chemical resistant” implies a substantial lack of reactivity or a substantial chemical inertness.

Materials that can be suitably employed as the nanofiltration membrane in the separation process of this invention include, without limitation, polyimides, polyvinylidene fluorides (PVDF), polyacrylonitriles (PAN), polysulfones, polyether sulfones, sulfonated polysulfones, cellulosics (for example, cellulose acetate, cellulose nitrate, regenerated cellulose), polyether imides, aliphatic polyamides, polyether ether ketones (PEEK), and ceramics (for example, alumina, zirconia).

Most preferably, the nanofiltration membrane used in the process of this invention is selected from polyimide membranes having a molecular weight cutoff of about 400 Daltons and having a permeability of about 30 L²/m²-h; preferably, those such membranes also having a maximum pressure rating of between about 40 and 80 bar; and

more preferably, those such membranes also being wound in spiral modules, and being stable in toluene, xylene, ethyl acetate, methyl ethyl ketone, and hexane. A most preferred nanofiltration membrane for use in the process of this invention comprises Davison STARMEM™ 240 brand polyimide membrane.

In the membrane separation method of this invention, the nanofiltration membrane allows in the course of an operating cycle a substantial portion of the olefin metathesis products, unconverted reactant olefins, and optional solvent to pass through the membrane as a permeate. In this context, the phrase “a substantial portion of the olefin metathesis products, unconverted reactant olefins, and optional solvent” means greater than about 80 percent, and preferably, greater than about 90 percent, by weight, of the total weight of these materials in the metathesis reaction mixture is passed as a permeate. Likewise, the nanofiltration membrane retains a substantial portion of the metathesis catalyst and metathesis catalyst degradation products as a retentate. In this context, the phrase “a substantial portion of the metathesis catalyst and metathesis catalyst degradation products” means greater than about 80 percent, and preferably, greater than about 90 percent, and more preferably, greater than about 99 percent by weight of the total weight of the catalytic metal in the metathesis catalyst and, optionally, catalyst degradation product(s) is retained as a retentate. The amounts of catalytic metal in the retentate and permeate provide a good measure of the amounts of catalyst in the retentate and permeate, respectively. Any suitable modern analytical technique, such as neutron activation, can be used to determine the amounts of catalytic metal in the retentate and permeate. As mentioned previously, the stabilizing ligand and non-ligand additive may separate into the permeate or the retentate depending upon the specific materials and membrane used.

The temperature of the membrane separation process may be any operable temperature that is tolerated by the membrane and the components of the metathesis reaction mixture. Generally, it may be beneficial to elevate the separation temperature over that of the metathesis process temperature, because an increased separation temperature decreases viscosity and increases the flow properties of the reaction mixture. A separation viscosity of greater than about 0.5 centipoise and less than about 10 centipoise, and preferably, less than about 5.0 centipoise, is preferred. Any temperature providing for the aforementioned viscosity range may be suitably employed.

The flow of the metathesis reaction mixture through the membrane is aided by increased pressure. Any operable pressure that aids in the separation may be suitably applied, although the pressure is generally lower than pressures used in reverse osmosis. Typical pressures for the separation are greater than about 150 psig (1050 kPa), and preferably, greater than about 200 psig (1400 kPa). Typical pressures are less than about 400 psig (2800 kPa), and preferably, less than about 300 psig (2100 kPa).

When the separation is effected as noted hereinabove on a metathesis reaction mixture, then a separation is achieved in which the metathesis catalyst is retained as a retentate and the olefin metathesis products, unconverted reactants, and optional solvent are obtained as a permeate. In one engineering embodiment, the separation may be effected in a separation unit distinct from the metathesis reactor unit. In such an embodiment the metathesis reaction mixture is transported in whole to the separation unit, or alternatively, a portion of the metathesis reaction mixture is continuously withdrawn from the metathesis reaction unit to the separation unit. The retentate containing the metathesis catalyst is generally recycled directly back to the metathesis reactor with or without treatment to remove catalyst degradation products. Typically, the permeate is separated by conventional methods (such as, fractional distillation, extraction, crystallization, chromatography, or some combination of these and/or other conventional methods) to recover the olefin product(s), and the resulting permeate containing unconverted reactants and optional solvent, essentially absent olefin product(s), is recycled to the metathesis process reactor. The combined reactor-separator design can be employed as a continuous loop.

In another engineering embodiment, the metathesis process can be conducted in a membrane reactor wherein a portion of the reactor wall is itself fabricated from a nanofiltration membrane. Suitable reactors of this type are described in WO 02/100528 and in J. M. van de Graaf et al., Chemical Engineering Science, 54 (1999) pp. 1441-1445, incorporated herein by reference. In this design, the metathesis reaction mixture is continuously flowed over the membrane wall of the reactor for simultaneous separation of olefin products, unconverted reactant olefins, and optional solvent. In such a continuous process, as a portion of the partially-converted reaction mixture is removed through the nanofiltration membrane wall, a fresh or recycle feed comprising olefin reactants, solvent, optional stabilizing ligand, and optional non-ligand additive are typically fed to the reactor.

The following examples are provided to illustrate the invention described herein, but should not be construed to limit the invention in any manner. In light of the disclosure herein, those of skill in the art will recognize modifications of the following illustrative embodiments that fall within the scope of the invention.

### EXAMPLE 1

A disk (76 mm diameter) was cut from a sheet of nanofiltration membrane (Koch MPF-50 SelRo™ brand polyvinylidene fluoride membrane) and inserted into a clean Millipore™ solvent-resistant stirred cell set on top of a membrane support plate. The stirred cell provided 40 cm² of effective filtering surface. The apparatus was operated on top of a magnetic stirrer, which rotated an impeller inducing flow tangential to the membrane surface. The membrane had been stored in a solution of ethanol and water (50:50 vol/vol). After assembling the stirred cell and securing its cover, the membrane disk was wetted in place with ethanol introduced through a filler cap, then blown partially clear with nitrogen to verify permeability and to remove excess ethanol. Nitrogen was passed through the cell for 30 minutes to form an inert atmosphere. Nitrogen flow was sustained throughout the filling procedure to blanket liquids in the cell.

Methyl oleate (Witco, 50 ml, 42.3 g) was transferred via cannula under nitrogen through a filler port to the stirred cell containing the polyvinylidene fluoride membrane. Fifty milliliters (50 ml) was considered to be the minimum volume needed to immerse the stirrer sufficiently and induce tangential flow. A toluene solution (0.665 g) containing Grubbs I ruthenium metathesis catalyst, namely, benzylidene-bis(tricyclohexyl-phosphine)dichlororuthenium, at a total ruthenium concentration of 3,975 parts per million (ppm) was injected under nitrogen blanket inside the stirred cell, thereby producing a feed concentration of 52.4 ppm Ru. The cell was operated under constant trans-membrane pressure (the pressure drop across the membrane and its support plate) by controlling the pressure applied to liquid at the top of the cell through the pressure inlet port. Permeate was collected at atmospheric pressure. The separation was conducted at 20° C.

The stirred cell was sealed after loading with methyl oleate and catalyst. Applied nitrogen pressure was increased to 60 psig (420 kPa) and the stirrer speed set at 300 revolutions per minute (rpm) At 120 hours under such conditions, a permeate (6.8 g) was collected. Residual retentate (35.95 g) was drained from the stirred cell into a flask. Both permeate and retentate samples were analyzed by neutron activation to determine the concentration of ruthenium, which indicates the presence of catalyst. The ruthenium concentration in the permeate was 8.9±0.5 parts per million (ppm). The ruthenium concentration in the retentate was 61±3 ppm. In view of the analyses, the permeate was substantially depleted in metathesis catalyst, whereas the retentate substantially comprised catalyst. Thus, 87 percent of the original supply of catalyst remained in the retentate.

### EXAMPLE 2

A disk (90-mm diameter) was cut from a sheet of STARMEM™ 240 polyimide nanofiltration membrane and placed on top of a membrane support plate inside a clean, empty high-pressure stirred cell (MET Ltd., 270 ml). The membrane had a rated cut-off of 400 Daltons and a permeability of 30 L/M²-h. The stirred cell provided 54 cm² of effective filtering surface. The cell operated on top of a magnetic stirrer, which rotated a magnetically coupled impeller inside the cell to induce flow tangential to the membrane surface.

After assembling the stirred cell and securing the bottom cover, a reagent-grade, liquid mixture of methyl oleate (50 mol percent), 1-decene (25 mol percent), and methyl 10-undecenoate (25 mol percent) was poured into the open top of the cell. The top cover was secured; pressure hoses were connected to a nitrogen cylinder, regulator, shut-off, and relief valves. Pressure (400 psig, 2,800 kPa) was applied to obtain a flow of catalyst-free solution through the membrane and thereby completely wet it while washing out any preservative liquid. After collecting roughly 60 ml of permeate from the membrane, gas pressure was blocked off and vented from the cell before opening the cell and draining free liquid.

A surrogate solution representing a metathesis reaction mixture was prepared by adding a metathesis catalyst complex, namely, [dichlorotris(triphenyl-phosphine)ruthenium(II)] (0.1635 g) to 500 ml (net weight 416.0 g) of the aforementioned liquid mixture made from methyl oleate, 1-decene and 10-undecenoate. The concentration of ruthenium in the resulting solution was 39 ppm, by weight. The solution was thoroughly mixed at 27° C., and approximately 270 ml of the solution was poured into the stirred cell. A sample was taken, designated (“FEED”), and set aside for measurement of Ru in the starting feed. The top cover of the cell was again secured in place along with connections to the regulated pressure source.

The magnetic stirrer was set at 400 rpm, and nitrogen pressure was gradually increased to 450 psig (3150 kPa) inside the sealed stirred cell when liquid emerged from the permeate tube. The separation was conducted at 20° C. Permeate was collected in a trace-clean bottle at atmospheric pressure after passing through the membrane. The net weight of permeate was recorded at regular intervals to determine local permeate flux. After 5 hours and 40 minutes, nitrogen was shut off to the cell and internal pressure was vented. Impeller speed was reduced to 350 rpm. The total weight of permeate collected equaled 180.2 g, and this sample was labeled “PERMEATE.” The top of the cell was removed and residual, concentrated solution containing catalyst, designated “RETENTATE,” was drained into a trace-clean bottle and weighed. Net weight of retentate equaled 17.69 g.

For an average density of mixed permeate equal to 0.8447 g/ml, the average permeate flux over the entire test equaled 6.97 liters/m²h (LMH).

One sample each of the starting feed, permeate, and retentate solutions were analyzed by neutron activation to determine the ruthenium concentration. The results are reported in Table 1:

TABLE 1

| Sample I.D. | [Ru]^{1,2,3} |
|---|---|
| FEED | 32 ± 1 ppm |
| RETENTATE | 340 ± 10 ppm |
| PERMEATE | N.D. |
| PERMEATE-2 | 120 ppb ± 10 ppb |

¹ppm = parts per million by weight
²N.D. = not detectable at ≦1 ppm Ru
³ppb = parts per billion by weight

A second measurement for ruthenium in the permeate at higher sensitivity was obtained. The entire permeate sample was provided for that purpose and sample “PERMEATE-2” showed 120 parts per billion (ppb) ruthenium, by weight.

The results show that a nanofiltration membrane is suitably employed to separate a metathesis catalyst from a metathesis reaction mixture. The permeate comprises the olefin metathesis products, unconverted olefin reactants, and solvent, absent ruthenium metathesis catalyst (less than 1 ppm). The retentate comprises the ruthenium metathesis catalyst.

What is claimed is:

1. A process for separating a homogeneous metathesis catalyst and optionally one or more homogeneous metathesis catalyst degradation products from a metathesis reaction mixture comprising contacting with a nanofiltration membrane a metathesis reaction mixture comprising, in addition to said homogeneous metathesis catalyst and said one or more optional homogeneous metathesis catalyst degradation products, one or more olefin metathesis products, one or more unconverted olefin reactants, and optionally a solvent, so as to allow a substantial portion of said one or more olefin metathesis products, said one or more unconverted olefin reactants, and said optional solvent to pass through the membrane as a permeate, while substantially rejecting said homogeneous metathesis catalyst and optionally said one or more homogeneous metathesis catalyst degradation products as a retentate.

2. The process of claim 1 wherein the metathesis reaction mixture is obtained by homo-metathesis, cross-metathesis, ring-closing metathesis, or ring-opening metathesis polymerization.

3. The process of claim 1 wherein the one or more olefin reactants each comprise a C₂₋₅₀ substituted or unsubstituted olefin.

4. The process of claim 3 wherein the one or more olefin reactants comprise an unsaturated fatty acid or unsaturated fatty acid ester.

5. The process of claim 1 wherein the metathesis reaction mixture is obtained from the cross-metathesis of a C₆₋₅₀ unsaturated fatty acid or C₆₋₅₀ unsaturated fatty acid ester with a C₂₋₁₀ olefin.

6. The process of claim 5 wherein the metathesis reaction mixture is obtained from the cross-metathesis of methyl oleate with ethylene, propylene, or decene.

7. The process of claim 1 wherein the metathesis catalyst comprises a transition metal selected from ruthenium, molybdenum, tungsten, rhenium, or a mixture thereof.

8. The process of claim 7 wherein the transition metal is ruthenium, and wherein the metathesis catalyst comprises ruthenium, two anionic ligands, two electronically neutral ligands, and one carbene ligand.

9. The process of claim 8 wherein the metathesis catalyst has the following formula: 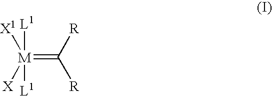
wherein M is ruthenium; each R is independently selected from hydrogen or a hydrocarbon radical selected from the group consisting of C₂-C₂₀ alkenyl, C₂-C₂₀ alkynyl, C₁-C₂₀ alkyl, C₆₋₂₅ aryl, C₁-C₂₀ carboxylate, C₂-C₂₀ alkoxy, C₂-C₂₀ alkenyloxy, C₂-C₂₀ alkynyloxy, C₆₋₂₅ aryloxy, C₂-C₂₀ alkoxycarbonyl, C₁-C₂₀ alkylthio, C₁-C₂₀ alkylsulfonyl, and C₁-C₂₀ alkylsulfinyl; X and X¹ are independently selected from chloride, bromide, and iodide; and each L¹ is independently selected from phosphine, sulfonated phosphine, phosphite, phosphinite, phosphonite, arsine, stibine, ether, amine, amide, sulfoxide, carboxyl, nitrosyl, pyridine, and thioethers.

10. The process of claim 1 wherein the metathesis catalyst is selected from the group consisting of dichloro-3,3-diphenylvinylcarbene-bis(tricyclohexylphosphine)ruthenium (II), bis(tricyclohexylphosphine)benzylidene ruthenium dichloride, tricyclohexylphosphine[1,3-bis(2,4,6-trimethylphenyl)-4,5-dihydroimidazol-2-ylidene][benzylidene]ruthenium (IV) dichloride, tricyclohexylphosphine[1,3-bis(2,4,6-trimethylphenyl)-4,5-dihydroimidazol-2-ylidene][benzylidene]ruthenium (IV) dibromide, tricyclohexylphosphine[1,3-bis(2,4,6-trimethylphenyl)-4,5-dihydroimidazol-2-ylidene][benzylidene]ruthenium (IV) diiodide, and the chelated ruthenium complexes represented by formula: 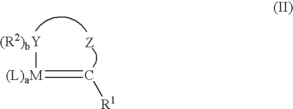
wherein M is Ru; each L is independently selected from neutral and anionic ligands in any combination that balances the bonding and charge requirements of M; a is an integer, preferably from 1 to about 4, which represents the total number of ligands L; R¹ is selected from hydrogen, straight-chain or branched alkyl, cycloalkyl, aryl, and substituted aryl radicals; Y is an electron donor group of an element from Group 15 or 16 of the Periodic Table; each R² is independently selected from hydrogen, alkyl, cycloalkyl, aryl, and substituted aryl radicals sufficient to satisfy the valency of Y; b is an integer, preferably 0 to about 2, representing the total number of R² radicals; and Z is an organic diradical that is bonded to both Y and the carbene carbon (C) so as to form a bidentate ligand, which ligand in connection with the M atom forms a ring of from about 4 to about 8 atoms.

11. The process of claim 10 wherein each L is independently selected from the group consisting of halides, cyanide, thiocyanate, phosphines of the formula PR³₃, amines of the formula NR³₃, water and ethers of the formula OR³₂, thioethers of the formula SR³₂, and ligands having the formulas: 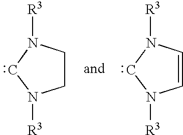
wherein each R³ in any of the aforementioned formulas is independently selected from the group consisting of hydrogen, C₁₋₁₅ alkyl; C₃₋₈ cycloalkyl; C₆₋₁₅ aryl and substituted aryl; and Z is selected from the following diradicals: ethylene (V), vinylene (VI), phenylene (VII), substituted vinylenes (VIII), substituted phenylenes (IX), naphthylene (X), substituted naphthylenes (XI), piperazindiyl (XII), piperidiyl (XIII): 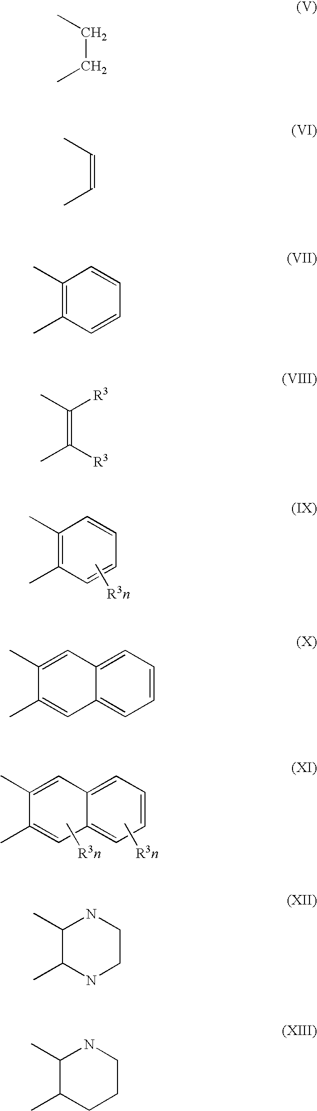
wherein each R³ is selected from hydrogen, C₁₋₁₅ alkyl; C₃₋₈ cycloalkyl; and C₆₋₁₅ aryl radicals; and wherein each n is an integer from 1 to about 4.

12. The process of claim 1 wherein the metathesis catalyst has the formula: 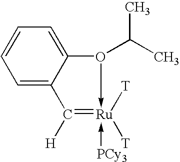
wherein each T is independently selected from Cl, Br or a mixture thereof; and PCy₃ represents tricyclohexylphosphine.

13. The process of claim 1 wherein the metathesis reaction mixture further comprises a stabilizing ligand, which is selected from the group consisting of tri(alkyl)phosphines, tri(aryl)phosphines, alkyldiarylphosphines, dialkylarylphosphines, ethers, phosphine oxides, phosphinites, phosphonites, phosphoramidites, pyridines, and any combination of the aforementioned compounds; optionally, in a molar ratio of stabilizing ligand to catalyst of greater than about 0.05/1 and less than about 2.0/1.

14. The process of claim 13 wherein the metathesis reaction mixture further comprises a stabilizing ligand, which is selected from tri(cyclohexyl)phosphine or tri(phenyl)phosphine.

15. The process of claim 1 wherein the metathesis reaction mixture further comprises a solvent selected from the group consisting of aromatic hydrocarbons, chlorinated aromatic hydrocarbons, alkanes, and chlorinated alkanes, ethers, and combinations thereof.

16. The process of claim 1 wherein the metathesis reaction mixture further comprises a non-ligand additive selected from the group consisting of phenolic compounds.

17. The process of claim 1 wherein a molar ratio of reactant olefin to metathesis catalyst in the metathesis reaction mixture ranges from greater than about 10:1 to less than about 10,000,000:1.

18. The process of claim 1 wherein the nanofiltration membrane has a molecular weight cutoff of greater than about 200 Daltons and less than about 800 Daltons.

19. The process of claim 1 wherein the nanofiltration membrane has a permeability of greater than about 1 L/m²-h of metathesis reaction mixture.

20. The process of claim 19 wherein the nanofiltration membrane has a permeability of greater than about 5 L/m²-h to less than about 100 L/m²-h of metathesis reaction mixture.

21. The process of claim 1 wherein the nanofiltration membrane is selected from the group consisting of polyimides, polyvinylidene fluorides (PVDF), polyacrylonitriles (PAN), polysulfones, polyether sulfones, sulfonated polysulfones, cellulosics, polyether imides, aliphatic polyamides, polyether ether ketones (PEEK), and ceramics.

22. The process of claim 21 wherein the nanofiltration membrane is selected from a polyimide having a molecular weight cutoff of about 400 Daltons and a permeability of about 30 L/m²-h of toluene at 30 bar; and optionally, having a maximum pressure rating of from 40 to 80 bars.

23. The process of claim 1 wherein the viscosity of the metathesis reaction mixture during the separation process ranges from greater than about 0.5 centipoise to less than about 10 centipoise.

24. The process of claim 1 wherein the pressure ranges from greater than about 150 psig (1050 kPa) to less than about 400 psig (2800 kPa).

25. The process of claim 1 wherein greater than about 80 weight percent of the total weight of the one or more olefinic metathesis products, the one or more olefin reactants, and the optional solvent is passed through the membrane as a permeate.

26. The process of claim 1 wherein greater than about 80 weight percent of the total weight of metal in the metathesis catalyst and said optional one or more metathesis catalyst degradation products is retained in the retentate.

27. A continuous metathesis-separation process comprising (a) contacting continuously one or more reactant olefins with a homogenous metathesis catalyst, optionally in the presence solvent, in a reactor under reaction conditions sufficient to prepare a metathesis reaction mixture comprising one or more product olefins that are different from the reactant olefins, one or more unconverted reactant olefins, the homogeneous metathesis catalyst, optionally solvent, and optionally one or more homogeneous metathesis catalyst degradation products; and (b) continuously contacting a portion of the metathesis reaction mixture with a nanofiltration membrane under conditions sufficient to allow a substantial portion of said one or more olefin metathesis products, said one or more unconverted olefin reactants, and said optional solvent to pass through the membrane as a permeate, while substantially rejecting said homogeneous metathesis catalyst and said optional one or more homogeneous metathesis catalyst degradation products as a retentate; (c) recycling said retentate comprising said homogeneous metathesis catalyst to process step (a); and (d) separating said olefin metathesis products from the permeate and recycling the resulting permeate comprising said one or more unconverted reactant olefins and optional solvent, essentially absent said olefin metathesis products, to process step (a).
